(19) United States
(12) Patent Application Publication
(10) Pub. No.: US 2014/0007870 A1
(43) Pub. Date: Jan. 9, 2014

(54) DEVICE AND METHOD FOR SUPPLYING AND DOSING GAS TO A BREATHING PERSON

(71) Applicants: Oskar Frånberg, Tumba (SE); Åke Larsson, Jarfalla (SE); Peter Lindholm, Solna (SE); Fredrik Frånberg, Tumba (SE)

(72) Inventors: Oskar Frånberg, Tumba (SE); Åke Larsson, Jarfalla (SE); Peter Lindholm, Solna (SE); Fredrik Frånberg, Tumba (SE)

(21) Appl. No.: 14/006,943

(22) PCT Filed: Mar. 19, 2012
(86) PCT No.: PCT/SE2012/050298

§ 371 Date: Sep. 23, 2013

Related U.S. Application Data
(60) Provisional application No. 61/466,956, filed on Mar. 24, 2011.

(30) Foreign Application Priority Data
Mar. 24, 2011 (SE) 1150260-6

(51) Int. Cl. A61M 16/20; A61M 16/12

(52) U.S. Cl. CPC A61M 16/208; A61M 16/12
USPC 128/203.12; 128/204.23

(57) ABSTRACT

A dosing device for supplying a gas, preferably oxygen, to a breathing person under physical strain and/or during recovery from physical strain, which dosing device is portable, and includes: a gas conduit comprising a connector configured to connect to a gas supply, a gas nozzle adapted to be held in front of the mouth and/or nose of the breathing person; a breathing cycle detector configured to detect the occurrence of inhalation and/or exhalation phase and exhalation phase in the breathing cycle; a gas flow controller, and a control unit configured to control the gas dosage supplied to the person dependent on values obtained from the breathing cycle detector, so that gas is supplied during the inhalation phase of the breathing cycle at a point in time and for a time period.

### TECHNICAL FIELD

The present invention relates to a device and a method for dosing gas to a person.

### BACKGROUND ART

Intense exercise is a way to achieve increased fitness. However, there is a individual limit for each person on how much oxygen can be supplied by the respiration system of the human body. By supplying additional oxygen, the person can sustain a higher workload while exercising and thus enhance his/her fitness. To enhance the result, oxygen is preferably supplied dependent on the physical strain. Methods for supplying oxygen to exercising persons are known in the art. For example, as disclosed in SE429926B an air stream nozzle can be attached to exercising equipment in front of the users face, and oxygen is supplied dependent on the load of the equipment.

### SUMMARY OF THE INVENTION

Devices for supplying oxygen hitherto available typically have relatively high oxygen consumption, and thus require large gas supplies. These devices are therefore difficult to carry along, since the gas source will be a large and heavy gas tube, a Liquid Oxygen (LOX) container, or an oxygen concentrator. There is a need for an improved device for supplying oxygen and/or other gases to a breathing person, having a low weight and low gas consumption.

The invention thus relates to a dosing device for supplying a gas, preferably oxygen, to a breathing person under physical strain and/or during recovery from physical strain. The device is portable, and includes a gas conduit comprising a means for connection to a gas supply; a gas nozzle adapted to be held in front of the mouth and/or nose of the breathing person; a breathing cycle detecting means; a means for measuring the physiological state of the breathing person; and a gas flow control means, and a control unit (7) for controlling the gas dosage supplied to the person dependent on values obtained from the breathing cycle detecting means, so that gas is supplied during the inhalation phase of the breathing cycle. Thereby, gas can be supplied only when effective to enhance the exercise effect, and only during a predetermined period of the breathing cycle. Thus, less oxygen will be needed, and substantially all oxygen supplied will be inhaled by the breathing person, leading to decreased oxygen utilization from the oxygen source as compared to using a freeflowing technique.

The dosing device may further include a means for measuring the physiological state of the breathing person, and means for setting a range of physiological state threshold values for gas supply, wherein the control unit is adapted to control the gas dosage so that gas is supplied to the breathing person only when the values obtained from the means for measuring the physiological state are within the range of physiological state threshold values, which corresponds to a certain physiological state. Advantageously, gas can be supplied during the first 50% of the duration of the inhalation phase of the breathing cycle, preferably during the first 25% of the duration of the inhalation phase. The breathing cycle detecting means may be adapted to detect when a specific part of the breathing cycle occurs such as exhalation or inhalation.

The breathing nozzle of the dosing device may be a flexible tube. The dosing breathing nozzle can advantageously by attached so as to not come in contact with the person's mouth or nose, and is adapted to be placed in front of the mouth or the person.

The dosing device according can include a gas supply, and gas of the gas supply can be chosen from oxygen, nitrogen, nitric oxide, nitrous oxide, carbon dioxide, helium, or a combination thereof. The gas conduit of the dosing device may comprise means for connecting to an additional gas supply, for supplying an additional gas, as a mixture or as two separate dosages from the same or different breathing nozzles.

The means for measuring the physiological state of the person is a means for measuring any of—the heart rate; or end-expired CO₂-concentration, or transcutaneous CO₂-concentration; or blood oxygen saturation; or respiratory rate; or uterine contractions.

The dosing can further include a means for measuring feedback information from a separate equipment, such as the power output from an exercise bike or speed of a treadmill, or the speed according to e.g. a global positioning system, which may be used e.g. while running or skiing.

Preferably, gas is not supplied when the heart rate value of the breathing person is below a first value or above a second value, but can be supplied only when the heart rate value is between said first value and said second value, in order to minimize the gas consumption.

The dosing device can further comprise controls operable by a user, for setting values for a range of values obtained from the means for measuring the physiological state during which gas is to be supplied, and/or a gas dosage supplied during each breathing cycle, and/or a mixture of different gases.

The dosing device can also comprise an interface for communication with an external computing device. Additionally, the dosing device can comprise an interface between the gas supply and the dosing device, consisting of an accumulator, in order to be able to deliver a higher flow during periods.

If desired an air separation device can be used as gas supply, said separation device having one outlet higher in oxygen content and one outlet higher in nitrogen content, and wherein the dosing device is adapted to be connected either to one of the outlets, or alternatively to be connected to both outlets whereby the breathing person can alternate between dosing with higher oxygen concentration or with higher nitrogen concentration during a session of use, or the dosing device could be operated to automatically alternate between higher oxygen concentration and nitrogen concentration during a session of use.

The invention also relates to a method of supplying and dosing gas, preferably oxygen, to a breathing person under physical strain and/or during recovery from physical strain. Said method comprises the steps of:
detecting the breathing cycle;
determining when an inhalation begins;
optionally measuring the physiological state of the breathing person, to obtain a physiological state value, and determining if the physiological state value is within a predetermined range; and
supplying to the person dependent on values obtained from the breathing cycle detecting means, so that gas is supplied during the inhalation phase of the breathing cycle.

### DETAILED DESCRIPTION

The present invention is based on the principle that a gas is to be used for a certain purpose by an athlete, or other breathing person, during exercise or recovery from exercise, and should be supplied only at certain conditions and only during predetermined selected time periods, thereby minimising the gas consumption.

The dosing device of the invention is thus a device for supplying a gas to a person under physical strain and/or during recovery from physical strain. The device is particularly suitable for athletes, but also persons/person having respiratory difficulties or dysfunctions could find it useful. For the sake of simplicity the person using the device of the invention is hereinafter referred to as “the person”, and this expression is intended to include any breathing person on any level of exercise, who is using the device.

The control unit of the gas dosing device is preferably adapted to control the gas dosage so that gas is supplied to the breathing person when appropriate. Further, the breathing cycle detecting means is preferably adapted to detect the frequency of inhalation and exhalation, and the predetermined point in time at which gas is supplied to the person is at the beginning of the inhalation in each breathing cycle. The breathing cycle detecting means is also adapted to detect when exhalation occurs.

The breathing nozzle is advantageously a flexible tube, and is preferably attached to a headset, and is adapted to be placed in front of the mouth of the person.

The dosing device may further include a gas supply. The gas of the gas supply is chosen from oxygen, nitrogen, nitric oxide, nitrous oxide, carbon dioxide, helium, air or a combination thereof. Gas can also be produced by an air concentrator by concentrating the oxygen or the nitrogen in air and supplying it to the dosing device. The gas conduit may comprise means for connecting to an additional gas supply, for supplying an additional gas. When the apparatus is connected to a gas supply with a limited flow, an accumulator can be connected in between gas supply and the apparatus and thus be able to deliver periods of higher gas flows than the mean flow of the gas source. For gas sources where air is used and separated in to a oxygen rich and an oxygen lean gas flow, the apparatus can be connected to all gas feeds and exercise or recovery could be conducted by alternating in between by for instance hypoxic exercise and hyperoxic recovery or alternating in between exercise with hyperoxia and hypoxia or alternating between hyperoxia, hypoxia and normoxia (air) to test what the exercise capacity in air is. One or more gas sources can also gas supply one or several gas dosing devices, so that for instance one air concentrator can supply hyperoxic and hypoxic gas mixtures to a whole team of people training simultaneously.

The means for measuring the physiological state of the person is preferably a means for measuring any of: the heart rate or end-expired CO₂-concentration, or breathing rate or transcutaneous CO₂-concentration; or blood oxygen saturation of the person.

Advantageously, the dosing device can be set such that gas is not supplied when e.g. the heart rate value or uterine contraction of the breathing person is below a first chosen or appropriate value or above a second chosen or appropriate value, but is supplied only when the heart rate value is between said first level value and said second level value or if the physiological state is above or below a certain level in order to minimize the gas consumption.

Further, the dosing device preferably comprises controls operable by a user, for setting values for a range of values obtained from the means for measuring the physiological state during which gas is to be supplied, and/or the amount of gas dosage supplied during each breathing cycle, and/or the mixture of different gases. The dosing device may preferably comprise an interface for communication with an external computing device.

The invention also relates to a method of supplying and dosing gas, preferably oxygen, to a breathing person under physical strain and/or during recovery from physical strain. The method comprises the steps of detecting the breathing cycle; determining when an inhalation begins; measuring the physiological state of the breathing person to obtain a physiological state value; determining if the physiological state value is within a range Or if the physiological state is above or below a certain; and if the physiological state fulfils this; then supply a gas bolus at the beginning of the inhalation

The dosing device is portable which means that it has a sufficient low weight to allow a person to carry it along during exercising, or to bring along to a gym. It also means that the dosing of gas is effective enough so that only small gas volumes will be needed.

The dosing device is a unit housing a number of components, and is adapted to be brought along by the athlete during any type of exercise or other similar activity. The dosing device includes the following components, which will be described in detail below.
a gas conduit with means for connection to a gas supply,
a gas nozzle adapted to be held in front of the mouth and/or nose of the breathing person;
a breathing cycle detecting means;
a means for measuring the physiological state of the breathing person; and
a gas flow control means, and
a control unit for controlling the gas dosage supplied to the person dependent on values obtained from the breathing cycle detecting means and the means for measuring the physiological state, so that gas is supplied during the breathing cycle at a predetermined point in time and for a predetermined time period.

The gas conduit comprises a means for connection to a gas supply. The gas can be supplied with any known technology, including pressurized vessels, oxygen concentrators, LOX containers, or chemical means. One or more gases can be delivered depending on the desired goal with the exercise. One goal with the exercise can be to expose the person to low oxygen pressures, so called high altitude exercise. In this case Nitrogen, N2, or other inert gas such as helium or if oxygen is tripped out of the air to be delivered, is added to the breathing gas in order to reduce the inhaled oxygen fraction. Oxygen (O2) is used for increased exercise levels and thus improvement in fitness or it could be used for faster recovery after exercise.

Helium (He) may be used to improve breathing capacity of asthmatic/COPD persons. This is a non-exhaustive list. Other gases can also be used for various purposes. The gas conduit of the dosing device can comprise means for connecting to an additional gas supply, for supplying an additional gas, to allow for use of two or more gases can also be used in combination to obtain several effects simultaneously, for example the delivery of both CO2 and O2 to maintain the respiratory drive unchanged during the use of high oxygen fractions, or He and O2 in combination to improve breathing capacity during exercise in persons with impaired capacity for ventilation Nitric Oxide (NO) can be used for lung ventilation/perfusion issues. Nitrous oxide (N₂O) can be used to improve comfort for athletes and subjects not used to heavy exercise such as e.g. obese persons starting a new training scheme. Carbon dioxide (CO₂) can be used for CO₂ tolerance exposures.

The device can also be used at high altitude to increase oxygen delivered to a person as a means of countermeasures for the environmentally low oxygen concentration at altitude

If desired, a gas supply e.g. in the form of a gas tube can be included in the portable dosing device. Since the gas dosage of the dosing device is so efficient, a small light weight gas tube can be sufficient.

The device may include a gas nozzle or breathing nozzle adapted to be held in front of the mouth and/or nose of the person in order to deliver gas to the vicinity of the mouth of the person. The gas nozzle could also be combined with the means to detect breathing described below. By using a gas nozzle which is held close to the mouth, the person will conveniently get his oxygen supply, without problems like drooling, or spasms in the jaw. This is an advantage over for example masks or mouthpieces, which may be connected with such problems. The gas nozzle is preferably a flexible tube, as this will be comfortable for the user and will not cause chafes on the wearer's skin. Such flexible tube may be a tube ending, e.g. a flexible silicon hose. The breathing nozzle is preferably attached to a headset, which is adapted to be conveniently attached to the head or neck of the person, such that the gas nozzle can be placed in front of the mouth or the person,but the device could also be used with a full face mask, oronasal mask, bite mouthpiece or other delivery connected to the user.

The breathing cycle detecting means, to detect the phases of the breathing cycle (or ventilation rhythm) of the person. This could be implemented by measuring a pressure drop in a tube located close to the mouth of the person, or the temperature close to the mouth thus detecting the difference in inspired and expired breathing gases, or the gas flow close to the mouth for example using a heated wire or heated thermistor technology, or the thorax circumference, or the thoracic impedance changes, preferably a heated thermistor.

One way to measure the ventilation rhythm is to place a pressure sensor in front of the mouth. The breathing cycle detecting means may advantageously be adapted to detect when exhalation occurs, since it the exhaled air flow concentrated flow profile resulting in a higher pressure if measured in this flow the inhaled air flow and is thus easier to detect. Based on the frequency of exhalation, the point in time when inhalation occurs can be calculated.

The measuring of the physiological state of the person could be made for example by measuring the heart rate, either with glued surface electrodes and wires or by a wireless belt comprising surface skin electrodes, battery power source, ECG amplifying electronic circuitry, ECG detection as well as a radio transmitter transmitting the information that a heart activation occurred (i.e. the detection of the R-wave of the ECG QRS complex) to a near receiver which has heart rate calculating capability, the latter is preferably integrated into the dosing device. The exertion level can be thus be estimated by measuring the heart rate of the person, e.g. by means of a conventional heart rate meter band. The physiological state of the person could also be made by measuring the end-expired CO₂ concentrations, F_{ETCO2}, or the transcutaneous CO2 concentrations, Tc_{CO2}, to determine for example the anaerobic threshold where some persons will start hyperventilation, thus lowering their F_{ETCO2}. Another way of measuring the physiological state of the person could be to measure the peripheral oxygen saturation (blood oxygen saturation), S_{pO2} , for example by using a oximeter technology. The benefit with heart rate measurement is the availability and simplicity of the measurement technology, the performance of the measurement during exercise as well as the low level of annoyance it creates for the person. With the respiratory rate the physical state can be determined by using the breathing cycle detecting device.

In order to ensure that the desired gas flow is supplied to the person a gas flow control means is included in the device. The gas flow control means could be a flow controlling valve, for example an on/off valve, which is closed in its normal position, or a proportional valve. Means for determining the amount of gas delivered to the person may include a flow meter. A flow restrictor, such as a dosage valve limiting and/or controlling the gas flow may be arranged in the gas conduit. The amount of gas delivered will then be proportional to the opening time of the dosage valve. The amount will of course also depend on the pressure in the gas supply. Alternatively the dosage amount can be controlled using an uncomplicated on/off valve that controls the fast emptying of a volume (i.e. a capacitor) that is slowly filled with a limited gas flow from the gas source via a flow restrictor.

The gas dosage supplied to the person is controlled by means of a control unit dependent on values obtained from the breathing cycle detecting means and the means for measuring the physiological state, so that gas is supplied during the breathing cycle at a predetermined point in time and for a predetermined time period. The control unit is preferably adapted to control the gas dosage so that gas is supplied to the breathing person only within a predetermined range of values obtained from the means for measuring the physiological state, which corresponds to a certain physiological state.

Advantageously, gas is not supplied when the heart rate value of the breathing person is below a first value or above a second value, but is supplied only when the heart rate value is between said first value and said second value, or above or below a limit. Thereby, gas is supplied only when it is most effective for achieving the desired exercise result, and accordingly the gas consumption is minimized. In case the gas source is a gas tube, a rather small tube can then be used, which facilitates handling and transport of the dosing equipment.

The dosing device may suitable comprise controls operable by the user. Such controls can be used for setting values for a range of values obtained from the means for measuring the physiological state during which gas is to be supplied. For example, a control could be the setting of a threshold heart rate value above which the gas dosage is to be enabled. Further, the device can include controls for setting values gas dosage supplied during each breathing cycle, or for the mixture of different gases. For example, the settings could include one or more of the following: the flow rate of the gas to be supplied to the person, the delay between the detected start of a breath and the onset of gas addition, and the duration of the gas addition.

The breathing cycle detecting means is adapted to detect the frequency of inhalation and exhalation, and to influence the point in time at which gas is supplied to the person is at the beginning of the inhalation of the of each breathing cycle. By supplying increased oxygen levels in the inhaled air, but only during inspiration it can be ensured that a large part of the oxygen is inhaled into the lung and consumed, when the person exercises at certain exertion levels. This leads to less oxygen loss, since less oxygen is supplied during time periods when the person does not inhale. Moreover, this enables use of the device indoors, since it is important to minimise oxygen levels in closed spaces due to the fire risk.

The dosing device preferably further comprises an interface for communication with an external computing device, e.g. a personal computer. The dosing device could have a communication function allowing the export of data and import of settings from an external computing device. The communication could be with wires (RS232, USB, Ethernet, etc.) or wireless (WLAN, BlueTooth, GSM/GPRS, Zigbe (IEEE 802.15.4), ANT+, etc.). The external computing device (e.g. a PC) may include functions that enable analysis of the exercise session, data storage, control of settings and exercise schemes etc. Such an external computing device could manage several dosing devices belonging to different persons. Such external computing device or the dosing device itself could be connected to an internet based exercise community function.

The dosing device houses all components that are to be close to the person, and can be run independently from the external computing device. The dosing device can include a storage unit to store settings and data related to the person and his/hers performance. If desired the dosing device can include a display and keyboard or other control means It can also contain basic input/output devices allowing the person to set up a exercise scheme and to monitor the progress. The input devices could be for example keys or a touch screen. The output device could be for example a LED indicating that the gas delivery is active or a small graphical display.

The dosing device suitably contains logic to determine when the gas addition is to be initiated and stopped. This could for example be comparing the actual heart rate, HR, to a threshold and if the HR is greater than that start a gas delivery at the detection or estimation of a person inspiration. The dosing device can also include an interface to internet and peripheral applications and data storage.

The present invention also concerns a method of supplying and dosing gas, preferably oxygen, to a person under physical strain and/or during recovery from physical strain. In the method the breathing cycle is detected and it is determined when an inhalation begins. This can be done by the different means discussed above. Further, the physiological state of the breathing person is determined, to obtain a physiological state value. Different ways of determining the physiological state value are possible, as discussed above. When the physiological state of the breathing person has been determined, it is determined if the physiological state value is within a predetermined range, and if so a gas dose is supplied at the beginning of the inhalation

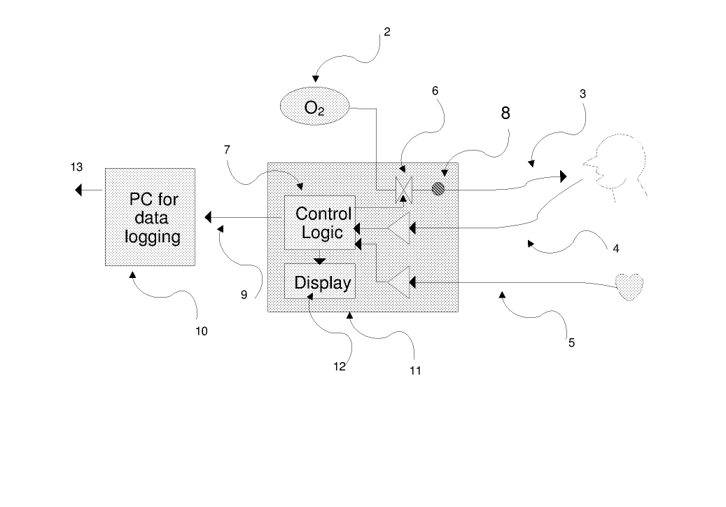

FIG. 1 shows an example of a dosing device according to the invention, which includes a means (4) to detect the phases of the breathing cycle of the person, and means (5) to detect the physiological state of the person. Means (3) to deliver gas is arranged in the vicinity of the mouth of the person. Means (8) to determine the amount of gas delivered to the person is also included. A source (2) of gas/gases to be delivered is connected to a gas conduit (1) of the device. A flow controlling valve (6) is arranged in the gas conduit. There is an interface (9) to an external computing device (i.e. a PC) (10). The dosing device includes an athlete unit (or user unit) (11) with display and keyboard (12), and gas addition control logic (7). An interface (13) enables contact to internet and peripheral applications and data storage.

1. A dosing device configured to supply a gas to a breathing person under physical strain and/or during recovery from physical strain, wherein the device is portable, and comprises:
a gas conduit comprising a connector configured to connect to a gas supply,
a gas nozzle adapted to be held in front of the mouth and/or nose of the breathing person;
a breathing cycle detector configured to detect the occurrence of inhalation and/or exhalation phase and exhalation phase in the breathing cycle;
a gas flow controller, and
a control unit configured to control the gas dosage supplied to the person dependent on values obtained from the breathing cycle detector, so that gas is supplied during the inhalation phase of the breathing cycle.

2. The dosing device according to claim 1, wherein the dosing device further includes a measuring component configured to measure the physiological state of the breathing person, and a gas supply controller configured to set a range of physiological state threshold values for gas supply, wherein the control unit is adapted to control the gas dosage so that gas is supplied to the breathing person only when the values obtained from the measuring component are within the range of physiological state threshold values, which corresponds to a certain physiological state.

3. The dosing device according to claim 1, wherein gas is supplied during the first 50% of the duration of the inhalation phase of the breathing cycle.

4. The dosing device according to claim 1, wherein the breathing cycle detector is adapted to detect when a specific part of the breathing cycle occurs.

5. The dosing device according to claim 1, wherein the breathing nozzle is a flexible tube.

6. The dosing device according to claim 1, wherein the breathing nozzle is attached so as to not come in contact with the person's mouth or nose, and is adapted to be placed in front of the mouth or the person.

7. The dosing device according to claim 1, further including a gas supply.

8. The dosing device according to claim 7, wherein the gas of the gas supply is selected from the group consisting of oxygen, nitrogen, nitric oxide, nitrous oxide, carbon dioxide, helium, and a combination thereof.

9. The dosing device according to claim 1, wherein the gas conduit comprises a connector configured to connect to an additional gas supply and to supply an additional gas, as a mixture or as two separate dosages from the same or different breathing nozzles.

10. The dosing device according to claim 1, wherein the measuring component is configured to measure a factor selected from the group consisting of
the heart rate; or
end-expired CO₂-concentration, or transcutaneous CO₂-concentration; or
blood oxygen saturation; or
respiratory rate; and
uterine contractions.

11. The dosing device according to claim 1, further including a monitor configured to measure feedback information from a separate piece of equipment.

12. The dosing device according to claim 1, wherein gas is not supplied when the heart rate value of the breathing person is below a first value or above a second value, but is supplied only when the heart rate value is between said first value and said second value, in order to minimize the gas consumption.

13. The dosing device according to claim 1, further comprising controls operable by a user, for setting values for:
a range of values obtained from the measuring component, during which gas is to be supplied, and/or
a gas dosage supplied during each breathing cycle, and/or
a mixture of different gases.

14. The dosing device according to claim 1, further comprising an interface for communication with an external computing device.

15. The dosing device according to claim 1, further comprising an interface between the gas supply and the dosing device, consisting of an accumulator, in order to be able to deliver a higher flow during periods.

16. The dosing device according to claim 1, wherein an air separation device is used as gas supply, said separation device having one outlet higher in oxygen content and one outlet higher in nitrogen content, and wherein the dosing device is adapted to be connected either to one of the outlets, or alternatively to be connected to both outlets whereby the breathing person can alternate between dosing with higher oxygen concentration or with higher nitrogen concentration during a session of use, or the dosing device could be operated to automatically alternate between higher oxygen concentration and nitrogen concentration during a session of use.

17. A method of supplying and dosing gas to a breathing person under physical strain and/or during recovery from physical strain, said method comprising the steps of:
detecting the breathing cycle;
determining when an inhalation begins;
optionally measuring the physiological state of the breathing person, to obtain a physiological state value, and determining if the physiological state value is within a predetermined range; and
supplying to the person dependent on values obtained from the breathing cycle detecting means, so that gas is supplied during the inhalation phase of the breathing cycle.

18. The dosing device according to claim 1, wherein the gas is oxygen.

19. The dosing device according to claim 1, wherein gas is supplied during the first 25% of the duration of the inhalation phase of the breathing cycle.

20. The method of claim 4, wherein the specific part of the breathing cycle is exhalation or inhalation.

21. The dosing device according to claim 11, wherein the feedback information is selected from the group consisting of a power output from an exercise bike, a speed of a treadmill, and a speed according to a global positioning system, which may be used while running or skiing.

22. The method of claim 17, wherein the gas is oxygen.
